(19) United States
(12) Patent Application Publication
(10) Pub. No.: US 2009/0043265 A1
(43) Pub. Date: Feb. 12, 2009

(54) METHOD AND APPARATUS FOR AUTO INJECTION OF A THERAPEUTIC

(76) Inventor: Robert M. Schneider, Tannersville, NY (US)

Correspondence Address: RAYMOND R. MOSER JR., ESQ.;MOSER IP LAW GROUP, 1030 BROAD STREET, 2ND FLOOR, SHREWSBURY, NJ, 07702, US

(21) Appl. No.: 12/186,850

(22) Filed: Aug. 6, 2008

Related U.S. Application Data
(60) Provisional application No. 60/964,242, filed on Aug. 10, 2007.

(51) Int. Cl. A61M 5/315

(52) U.S. Cl. 604/218

(57) ABSTRACT

Provided, among other things, is a therapeutic dispensing device (100) comprising: a handle (10) with proximal (44) and distal (46) end, defining cavity (11), and containing a window at the distal end (13); a channel (38) positioned within the cavity; a carriage (12) supported by the handle and configured to slide into the handle through the opening, and at least partially into the channel, the carriage having proximal end (2) and distal end (4), the proximal end having a first opening (8) and the distal end having a substantially non flexible engagement surface and a second opening (6); a syringe assembly (14) supported by the carriage and at least partially positioned in the channel, such assembly comprising a barrel (20) supporting plunger (16), and needle (24) for dispensing contents of the barrel; and a spring means (40) operably biased between the carriage and the handle.

### CROSS-REFERENCE TO RELATED APPLICATIONS

This application claims benefit of U.S. provisional patent application Ser. No. 60/964,242, filed Aug. 10, 2007, which is herein incorporated by reference.

### FIELD OF THE INVENTION

The present invention relates with methods and apparatuses of dispensing therapeutics, and more specifically, with methods and apparatus of dispensing therapeutics using an auto injection device.

### BACKGROUND

Frequently, when an individual suffers from a medical episode, such as severe allergic reaction of a bee sting, food ingestion, early signs of angina, etc., the medication, such as, for example, chemicals, therapeutics, preventive medicines, etc., that may save their life are not readily available or difficult to administer. During such episodes, any time wasted searching for the medication may be critical to the individual or ineffective administration may cause a life threatening situation.

Thus, in an emergency or if an individual believes they are suffering from the early signs of a medical problem, the individual is forced to manipulate a medical dispenser while enduring some degree of panic and perhaps physical pain.

Therefore, it would be highly desirable to have a self-contained therapeutic dispenser that supports a therapeutic is dispensed from the therapeutic dispenser and be accomplished in a facile manner.

Further, it is also required that in dispensing these therapeutics, the dispenser does not leave room for mishap, for example, leaving a needle projecting out of the device after use. Situations such as these pose problems, especially for an individual who has recently suffered from an emergency medical condition, and may not be fully alert. This is highly undesirable, because an uncovered needle may cause the individual to get hurt inadvertently or pose a danger of infection to others.

Accordingly, there exists a need for a self-contained therapeutic dispensing device which is employed in a facile manner. Further there exists a need for a therapeutic dispensing device that is easily activated to dispense a therapeutic via engagement of the therapeutic dispenser with a body surface of an individual. Further, there exists a need to provide a therapeutic dispensing device that overcomes all the above disadvantages, and provides the used device in an end configuration that is safe and easily stored or disposed.

Objects and advantages of the present disclosure are set forth in part herein and in part will be obvious therefrom, or may be learned by practice of the present disclosure, which is realized and attained by means of the instrumentalities and combinations pointed out in the appended claims. The apparatus and methods of the present disclosure consist of novel parts, constructions, arrangements, combinations, steps and improvements herein shown and described.

### SUMMARY

According to certain aspects, the present invention provides a therapeutic dispensing device comprises a handle defining a cavity, and a channel (38) positioned within the cavity with the distal end of the cavity having a window. A carriage (12) is supported by the handle and configured to slide into the handle through the opening, and at least partially into the channel. The proximal end of the carriage has a first opening and the distal end has a substantially non flexible engagement surface and a second pierceable opening. A syringe assembly (14) is further supported by the carriage and at least partially positioned in the channel and a spring means (40) is operably biased between the carriage and the handle.

According to certain other aspects, the present invention provides a method of use, in which the device is positioned such that the distal end of the carriage is held against a location on the user body and a force is applied, thereby causing the plunger to move downward into the barrel, causing the needle of the syringe to pierce through the second opening into the user body and dispensing the therapeutic into the user body. Releasing the force causes the spring means to bias the carriage such that the carriage extends and covers the previously exposed needle.

### BRIEF DESCRIPTION OF THE DRAWINGS

The foregoing and other advantages and features of the invention will become apparent upon reading the following detailed description and upon reference to the drawings in which:

FIG. 1 is a perspective view of a therapeutic dispensing device according to an embodiment of the present invention;

FIG. 2 is a exploded view of the cross section of the device of FIG. 1 with an initial configuration before use;

FIG. 3 is a top view of the device of FIG. 2 on an application of a force;

FIG. 4 is a top view of the device of FIG. 2 in a configuration in use;

FIG. 5 is a top view of the device of FIG. 2 in another configuration in use; and

FIG. 6 is a top view of the device of FIG. 2 in a configuration after use.

### DETAILED DESCRIPTION

As used herein, the term “a” refers to at least one, unless otherwise mentioned. As used herein, the term “include” refers to includes without limitation.

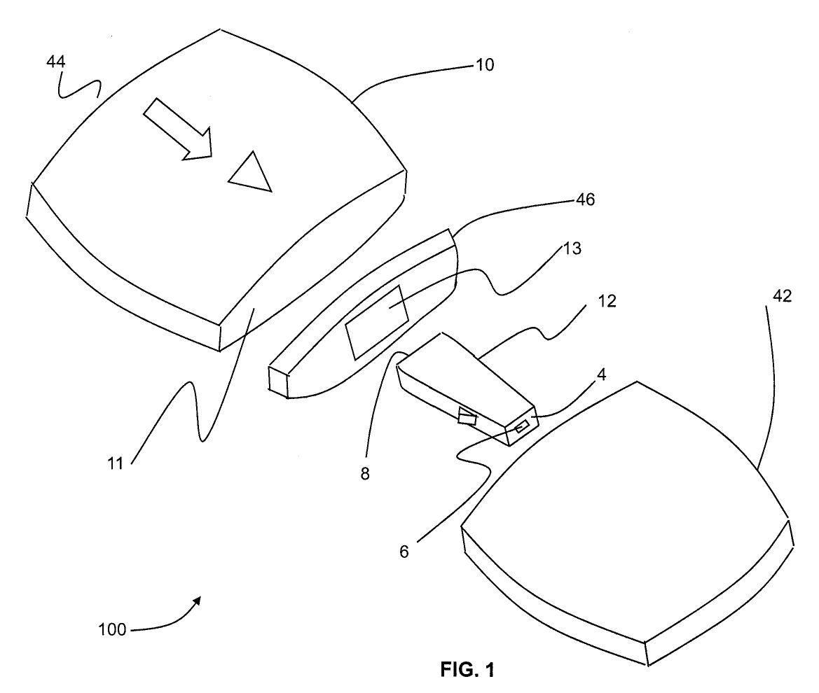

FIG. 1 illustrates a therapeutic dispensing device 100 according to an aspect of the present invention. The device 100 which includes a handle 10 defining a cavity 11. The handle 10 has a proximal end 44 and a distal end 46.

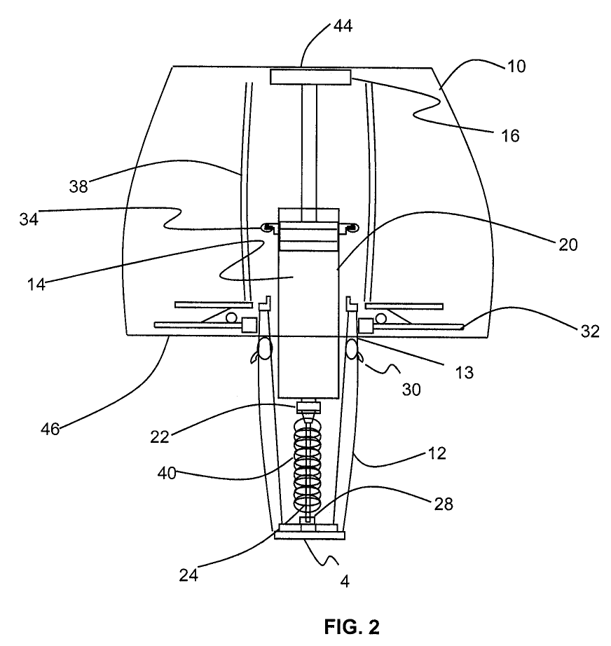

In FIG. 2 channel 38 is positioned within the cavity 11 (FIG. 1), and the distal end 46 contains a window 13. A carriage 12 is supported by the handle 10, and the carriage 12 is configured to slide into the handle 10 through the window 13. Further, the carriage 12 is configured to slide at least partially into the channel 38. The carriage 12 has a proximal end 2 and a distal end. The proximal end 2 has a first opening 8 (FIG. 1) and the distal end has a substantially non flexible engagement surface 4 with a second opening 6 (FIG. 1) in the distal end. The second opening 6 may be configured to be pierceable, for example, by a needle extending out of a device, and accordingly the second opening 6 may be covered with a pierceable film (not shown). A syringe assembly 14 is supported by the carriage 12 and further at least partially positioned in the channel 38. The syringe assembly 14 includes a barrel 20 configured to hold contents such as a therapeutic, a plunger 16 configured for pushing the contents of the barrel, and a needle 24 coupled to the barrel 20 through a needle hub 22. Further, a spring means 40 is positioned to be operably biased between the carriage 12 and the handle 10.

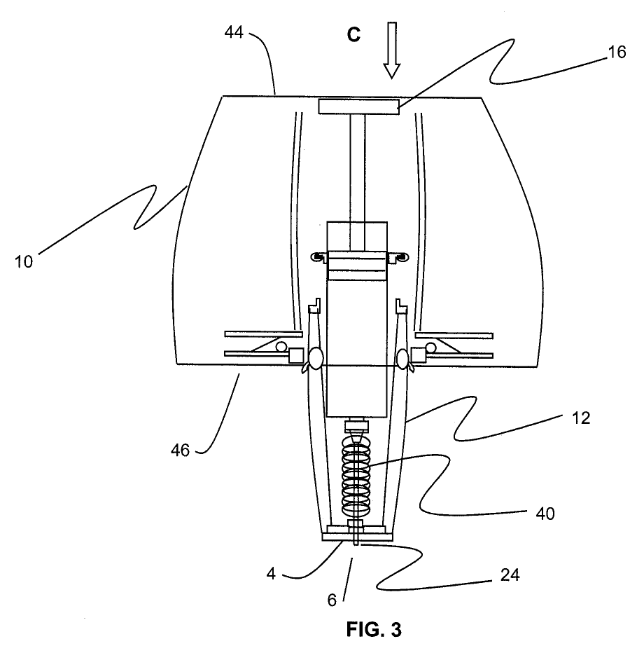

These elements are configured to form a stream lined assembly of the device 100, further having a cap 42 (FIG. 1). According to an embodiment, as shown in FIG. 2, the syringe assembly 40 is positioned such that the pusher 16 is rigidly engaged with the distal end 44 so that when the distal end 44 moves in the direction of engagement surface 4, the plunger 16 is accordingly pushed downwards. Further, the needle 24 portion of the syringe assembly 40 is positioned at least partially into the carriage 12, and the needle 24 is aligned to be extendable through the opening 6. The carriage 12 is positioned to be movable through the window 13 within the channel 38, while the syringe assembly 40 extends into the carriage 12 through the opening 8 (FIG. 1). The spring means 40 is positioned to be operably biased between the carriage 12 and the handle 10. According to an embodiment, the spring means 40 may include springs, such as one or more helical springs, torsion springs, leaf springs, elastic wires, among others, or a combination of these. In the embodiment illustrated in the figures, the spring means 40 is a helical spring 40 engaged between the needle hub 22 and a stopper 28. Accordingly, as shown in FIG. 3, the spring 40 biases the movement of the handle 10 relative to the carriage 12 in the direction as indicated by “C”. It is appreciated here that while in the present illustrations, the spring means 40 are engaged at the needle hub 22 and the stopper 28, the embodiments are not limited to engagement at these points. Those skilled in the art will readily appreciate various spring means configurations to bias the movement of the handle 10 relative to the carriage 12, in the manner described herein, and all such obvious variations are included within the scope of the claims appended hereto.

According to various embodiments, as shown in FIG. 3-6 the engagement surface 4 is configured to engage with a user body such that when a force “C” is applied to the proximal end 44 of the handle 10 in the direction of the distal end 46, the needle 24 of the syringe assembly 14 pierces the second opening 6. This causes the therapeutic to be dispensed from the barrel 20 through the needle 24 into the user body. During the process, the carriage 12 retracts inside the handle 10, or the handle 10 encloses the carriage 12, while the needle 24 projects out of the opening 6. Further, during this process, the spring 40 is compressed, and while the spring 40 is being compressed, the needle 24 starts extending outside the opening 6. Release of the force causes the spring 40 to decompress. Decompression of the spring 40 biases the carriage 12 to project outside of handle 10 and cover previously exposed needle 24

According to an embodiment, the auto injection device 100 as shown in FIG. 2, further includes a carriage arrestor 30 positioned on the carriage 12, a slidable clip 32 positioned in the distal end 46 portion of the handle 10 and configured to engage with the carriage arrestor 30. For example, FIG. 3 illustrates actuation of the slidable clip 32 on engagement with the carriage arrestor 30. The arrestor prevents the carriage from retract after use.

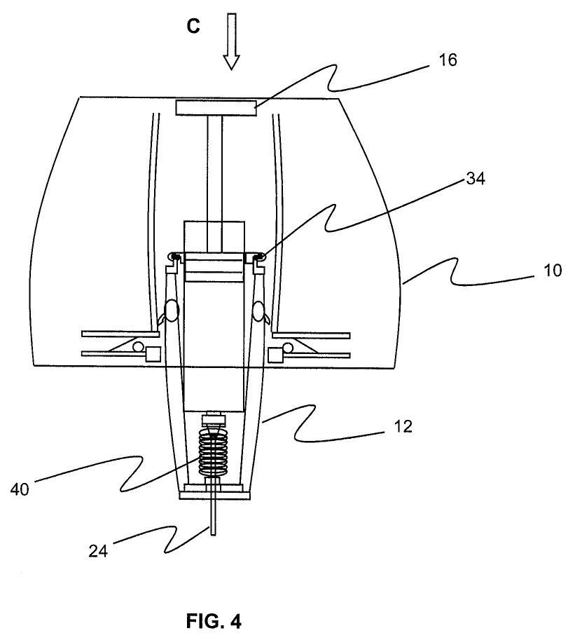

According to yet another embodiment, the auto injection device 100, further includes rotatable clips 34 configured to restrict the movement of the plunger until actuation of the clips 34. On actuation, the rotatable clips 34 are configured to release the plunger 16 for free movement. For example, as the embodiment of FIG. 4 illustrates, on engagement of the rotatable clips 34 with the carriage 12 proximal end 2, the rotatable clips 34 rotate about their axis, allowing the plunger 16 to move inside the barrel 20. According to the embodiment illustrated by the FIG. 4, the spring 40 reaches its maximum compression during use on actuation of the rotatable clip. It is appreciated that maximum compression during use may not be the maximum compression of the spring 40, and the spring may be further compressible. In the alternative, a flexible washer can be placed about the plunger. This would function to prevent actuation of the plunger until actual use.

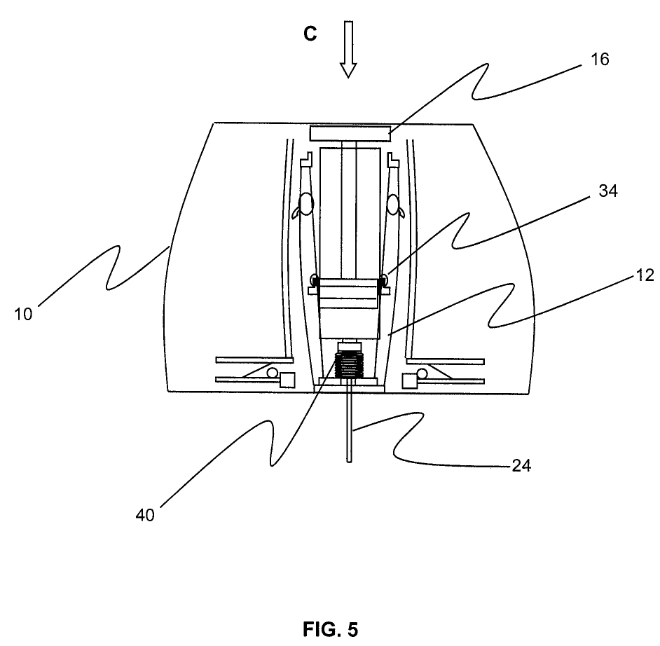

Further, as illustrated in FIG. 5, the handle 10 leads to actuation of the rotable clip 34 releasing the plunger 16, and on full displacement of the plunger 16 inside the barrel 20, the needle 24 extends outside the carriage 12 to its maximum extension outside the carriage 12 during use. It is appreciated here that the spring 40 may reach its maximum compression during use at instances other than when the needle 24 reaches its maximum extension outside the carriage 12. For example, according to the embodiments illustrated herein, the needle 24 reaches its maximum extension outside the carriage 12 at an instance after the spring 40 reaches its maximum compression during use. However, those skilled in the art will appreciate that this design aspect may be varied while still preserving all the functionalities and advantages of the present invention, and all such variations are included within the scope of the present claims.

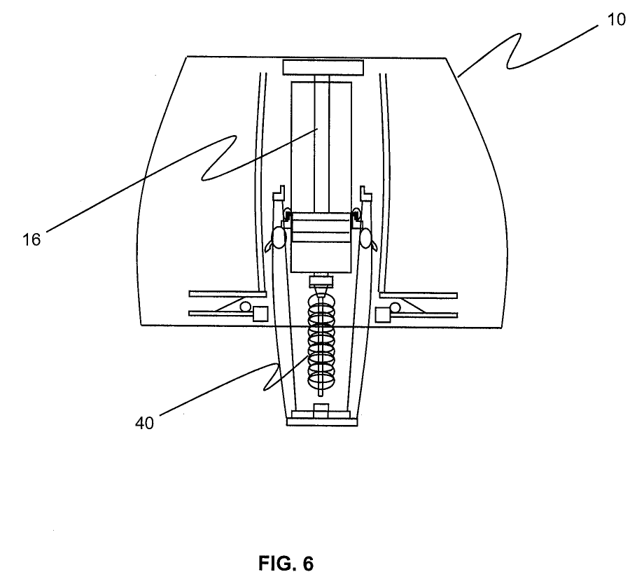

FIG. 6 illustrates the removal of force “C” from the device 100 (or the handle 10) and accordingly the spring is decompressed. In this configuration, carriage 12 projects outside of handle 10 and completely covers needle 24. The plunger 16 moves to its maximum displacement within the barrel 20, thereby decreasing the total syringe assembly 14 length, and accordingly, in the rest position, the carriage 12 projects out of the handle 10 with a length less than in the original configuration (FIG. 2). The cap 42 (FIG. 1) may then be replaced onto the handle 10 to store the used device in a safe configuration. It is appreciated here that the cap 42 is configured to be releasably engaged with the handle 10, using various known methods, such as click-lock mechanisms and the like, among various others.

According to various other embodiments, the device 100 may further include a medication holder (not shown) for storing additional therapeutics, such as, for example, an injectible therapeutic, a non-injectible therapeutic and the like. The cap 42 is configured to define a cavity configured to receive the handle 10, as well as configured to receive the medication holder having an additional therapeutic. While in certain embodiments, the medication holder may be positioned in the handle 10, in other embodiments the medication holder may be positioned in the cap 42 and the handle 10 may have a receiving cavity (not shown) for the medication holder.

According to another aspect of the present, a method of dispensing a therapeutic into a user body includes providing an auto injection device, such as, for example, the auto injection device 100, and positioning the distal end 4 of the carriage 12 against a location on the user body. Thereafter, a force “C” is applied, for example, by the user, on the proximal end 44 of the handle 10. It is noted here that the broad shape of the handle 10 is advantageously easy to grip and apply force to, especially in situations of emergency. For example, when a person has an allergic reaction, it may be relatively difficult to grip thinner or cylindrical objects. Such cylindrical devices can slip in a hand moist with nervous perspiration. In contrast, the handle 10 design illustrated herein is relatively easier to grip in the palm of the hand or folded fingers, and operate. There is no chance that the device will slip through the user's hand. Application of the force causes the plunger 16 to move downwards into the barrel 20. Accordingly, the needle 24 pierces through the second opening 6 into the user body and dispenses the therapeutic into the user body. After the therapeutic has been released into the user body, the force may be released, which causes the spring 40 to bias the carriage 12 such that the carriage 12 projects out of handle 10, and the needle 24 is completely covered by carriage 12.

In another aspect, the present invention provides a method of assembling an auto injection device, such as the device 100. The method includes providing a handle 10 defining a cavity 11 in 2 symmetric parts that snap together. The handle 10 has a proximal end 44 and a distal end 46, and the handle further includes a channel 38 positioned within the cavity 11. The distal end 46 of the cavity 11 contains a window 13. The method further includes providing a carriage 12 configured to be supported by the handle 10. The carriage 12 is configured to be slidable into the handle 10 through the window 13, and further, the carriage 12 is slidable at least partially into the channel 38. The carriage 12 has a proximal end 2 and a distal end 4. The proximal end 2 has a first opening 8 and the distal end has a substantially non flexible engagement surface 4. The engagement surface 4 has a second opening 6. The second opening 6 may be configured to be pierceable, for example, by a needle extending out of a device, and accordingly the second opening 6 may be covered with a pierceable film (not shown). The carriage 12 is positioned to reside within the channel 38 at least partially. Further, a syringe 14 is positioned at least partially in the channel 38, and the syringe assembly is configured to be supported by the carriage 12. The syringe assembly 14 includes a barrel 20, a plunger 16 configured for pushing the contents of the barrel, and a needle 24 coupled to the barrel 20 through a needle hub 22. Further the method includes providing a spring means 40 positioned to be operably biased between the carriage 12 and the handle 10. With such a device, any conventional pre-filled syringe of appropriate size can be snapped into this device to provide an inexpensive safety syringe.

The illustrated embodiments and other obvious variants intended to be included within the scope of the present invention provide various advantages. For example, the handle of the present invention is relatively easier to grip in the palm of the hand and hence provides ease of operation in instances of emergency, for example, an allergic reaction. Further, the carriage is configured to project and cover the needle, thereby making it safe to store and dispose off the device.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. A therapeutic dispensing device (100) comprising;
a handle (10) defining a cavity (11), the handle having a proximal end (44) and a distal end (46);
a channel (38) positioned within the cavity, wherein the distal end of the handle contains a window (13);
a carriage (12) supported by the handle, the carriage configured to slide into the handle through the opening, and at least partially into the channel, the carriage having a proximal end (2) and a distal end (4), the proximal end having a first opening (8) and the distal end having a substantially non flexible engagement surface and a second opening (6) therein;
a syringe assembly (14) supported by the carriage and at least partially positioned in the channel, the syringe assembly comprising a barrel (20) supporting a plunger (16), and a needle (24) for dispensing contents of the barrel; and
a spring means (40) operably biased between the carriage and the handle.

2. The therapeutic dispensing device of claim 1, further comprising:
a substantially non-flexible engagement surface (4) at the distal end of the carriage; and
a needle hub (22) in the syringe assembly, the needle hub having the needle (24) extensible through the second opening.

3. The therapeutic dispensing device of claim 2, wherein the engagement surface is configured to engage with a user body such that when a force is applied to the proximal end of the handle in the direction of the distal end of the handle, the needle of the syringe assembly pierces the second opening.

4. The therapeutic dispensing device of claim 3, wherein on application of a force on the proximal wall of the handle is configured to apply the force to the plunger causing the contents of the barrel to exit through the needle.

5. The therapeutic dispensing device of claim 4, wherein the release of the force is configured to cause the spring means to bias the carriage to return to its original position.

6. The therapeutic dispensing device of claim 1, further comprising:
a carriage arrestor (30) positioned on the carriage;
a slidable clip (32) positioned at a distal end (46) of the handle and configured to engage with the carriage arrestor;

7. The therapeutic dispensing device of claim 1, further comprising a rotable clip (34) configured to restrict the movement of the plunger, wherein the rotable clip is configured to release the plunger on engagement of the rotable clip with the carriage

8. The therapeutic dispensing device of claim 1, wherein on application of a force on the handle, the spring means is configured to compress and the carriage is configured to retract into the handle, and wherein the spring means reaches its maximum compression during use on actuation of the rotable clip.

9. The therapeutic dispensing device of claim 8, wherein on application of a force on the handle is configured to extend the needle outside the carriage to its maximum extension during use.

10. The therapeutic dispensing device of claim 9, wherein after dispensing the contents of the barrel and removal of the force, the spring is configured to decompress thereby retracting the needle into the carriage such that the carriage comes to rest at least partially out of the handle.

11. The therapeutic dispensing device of claim 1, wherein the handle includes at least one medication holder for storing additional therapeutics.

12. The therapeutic dispensing device of claim 1, further comprising a cap (42) to enclose the carriage and the handle, and wherein the cap cooperates to define a holder configured to support an additional therapeutic.

13. A method of dispensing a therapeutic into a user body, the method comprising:
providing a therapeutic dispensing device comprising
a handle (10) defining a cavity, the handle having a proximal end (44) and a distal end (46),
a channel (38) positioned within the cavity, wherein the distal end of the cavity contains a window,
a carriage (12) supported by the handle, the carriage configured to slide into the handle through the opening, and at least partially into the channel, the carriage having a proximal end and a distal end, the proximal end having a first opening and the distal end having a substantially non flexible engagement surface and a second pierceable opening therein,
a syringe assembly (14) supported by the carriage and at least partially positioned in the channel, the syringe assembly comprising a barrel (20) supporting a plunger (16), and a needle for dispensing contents of the barrel, and
a spring means (40) operably biased between the carriage and the handle;
positioning the distal end of the carriage against a location on the user body;
applying a force on the proximal end of the handle thereby causing the plunger to move downward into the barrel, causing the needle of the syringe to pierce through the second opening into the user body and dispensing the therapeutic in to the user body; and
releasing the force causing the spring means to bias the carriage such that the carriage projects out of the handle and completely covers the needle.

14. A method of assembling a therapeutic dispensing device, the method comprising:
providing a handle (10) defining a cavity, the handle having a proximal end (44) and a distal end (46), the handle comprising a channel (38) positioned within the cavity, wherein the distal end of the cavity contains a window;
providing a carriage (12) configured to be supported by the handle, the carriage configured to slide into the handle through the opening, and at least partially into the channel, the carriage having a proximal end and a distal end, the proximal end having a first opening and the distal end having a substantially non flexible engagement surface and a second pierceable opening therein, wherein the carriage is positioned to reside within the channel;
positioning a syringe assembly (14) at least partially in the channel, the syringe assembly configured to be supported by the carriage, and the syringe assembly comprising a barrel (20) supporting a plunger (16), and a needle for dispensing contents of the barrel; and
providing a spring means (40) to be operably biased between the carriage and the handle.
